(19) United States
(12) Patent Application Publication
(10) Pub. No.: US 2018/0055355 A1
(43) Pub. Date: Mar. 1, 2018

(54) Systems and Methods for Angiography and Motion Corrected Averaging

(71) Applicants: Marinko Venci Sarunic, Burnaby (CA); Morgan Lindsay Heisler, Maple Ridge (CA); Myeong Jin Ju, Vancouver (CA); Yifan Jian, Burnaby (CA); Mirza Faisal Beg, Coquitlam (CA); Arman Athwal, Delta (CA); Sieun Lee, Vancouver (CA)

(72) Inventors: Marinko Venci Sarunic, Burnaby (CA); Morgan Lindsay Heisler, Maple Ridge (CA); Myeong Jin Ju, Vancouver (CA); Yifan Jian, Burnaby (CA); Mirza Faisal Beg, Coquitlam (CA); Arman Athwal, Delta (CA); Sieun Lee, Vancouver (CA)

(73) Assignee: Marinko Venci Sarunic, Burnaby (CA)

(21) Appl. No.: 15/792,171

(22) Filed: Oct. 24, 2017

Related U.S. Application Data
(63) Continuation-in-part of application No. PCT/US16/51369, filed on Sep. 12, 2016.
(60) Provisional application No. 62/411,854, filed on Oct. 24, 2016.
(60) Provisional application No. 62/411,881, filed on Oct. 24, 2016.
(60) Provisional application No. 62/217,508, filed on Sep. 11, 2015.

(51) Int. Cl. A61B 3/12; A61B 5/00; A61B 3/10

(52) U.S. Cl. CPC A61B 3/1233; A61B 3/1241; A61B 3/0058; A61B 3/102; A61B 5/7203

(57) ABSTRACT

Optical coherence tomography (OCT) may be used to acquire cross-sectional or volumetric images of any specimen, including biological specimens such as the retina. Additional processing of the OCT data may be performed to generate images of features of interest. In some embodiments, these features may be in motion relative to their surroundings, e.g., blood in the retinal vasculature. In some embodiments, an acquired image may be degraded by motion artifact. The proposed invention describes OCT system embodiments that may be configured for multi-scale imaging, with the capability of switching between low or high lateral resolution, and with the assistance of adaptive optics for aberration correction. The invention also describes a method for enhancing OCT image quality by reducing or eliminating the negative effects introduced by sources and speed by performing bidirectional scanning. The proposed invention describes a method for the combination of images acquired by OCT for the automatic registration and averaging of features, such as blood vessels in images acquired with OCT angiography.

### CROSS REFERENCE TO RELATED APPLICATIONS

The instant application is a utility application and claims priority to the pending U.S. provisional patent applications: No. 62/411,854 titled “Method for Strip-based Motion Corrected Averaging of Retinal Angiography Images.”, filed on Oct. 24, 2016; and No. 62/411,881 titled “Method for phase stabilized flow contrast retinal angiography.”, filed on Oct. 24, 2016. The entire disclosure of the Provisional U.S. patent Application Nos. 62/411,854 and 62/411,881 are hereby incorporated by this reference in its entirety for all of its teachings. This benefit is claimed under 35. U. S. C. § 119. The instant application is a continuation-in-part application and claims priority to the pending PCT/US 2016/051369 titled “Coherence-Gated Wavefront-Sensorless Adaptive-Optics Multi-Photon Microscopy” filed on Sep. 12, 2016. The entire disclosure of the PCT/US 2016/051369 is hereby incorporated by this reference in its entirety for all of its teachings.

### FIELD OF TECHNOLOGY

The description is relevant to imaging of biological specimen such as a retina using a form of low coherence interferometry such as optical coherence tomography (OCT) and optical coherence domain reflectometry (OCDR). Embodiments of this invention relate to acquisition of high resolution images, flow contrast images, or the registration and averaging of these images.

### BACKGROUND

Optical coherence tomography (OCT) provides cross-sectional images of any specimen including biological specimens such as the retina with exquisite axial resolution, and is commonly used in ophthalmology. OCT imaging is an important aspect of clinical care. In ophthalmology, it is used to non-invasively visualize the various retinal structures to aid in a better understanding of the pathogenesis of vision-robbing diseases.

Extensions to conventional OCT imaging have been developed for enhancing visualization of the blood circulation, also referred to as angiography. The resulting image data is information rich, and requires proper analysis to assist with screening, diagnosis, and monitoring of retinal diseases.

The noninvasive, in vivo visualization of the retinal microvasculature using OCT-A can be instrumental in studying the onset and development of retinal vascular diseases. Quantitative measurements, such as capillary density, can be used to stratify the risk of disease progression, visual loss, and for monitoring the course of diseases. Due to various forms of artefact and poor contrast, it is often difficult to trace individual vessels in this layer when only one en face image is visualized. An additional challenge to this end is the small dimension and pulsatile flow of the retinal capillaries, making them less consistently visible and difficult to distinguish from the speckle noise relative to larger vessels. This limits the detection sensitivity for changes in the retinal microvascular circulation due to diseases, aging, or treatment. Methods for reliable visualization of the microvasculature in the OCT-A images are required for studies conducting longitudinal and cross-sectional quantitative analysis.

### SUMMARY

The invention discloses systems and methods for the acquisition and automated registration and averaging of serially acquired OCT images. In one embodiment, the OCT system comprises a beam splitter dividing the beam into sample and reference paths, light delivery optics for reference and sample, a beam combiner to generate optical interference, a detector to convert the signal into electronically readable form, and a processor for controlling the acquisition of the interference signal and generating images. In one embodiment of the invention, the OCT light delivery optics to the sample are configured for high lateral resolution imaging including adaptive optics. In one embodiment of the invention, an additional stable interferometric signal is used for phase stabilization of the OCT signal. In one embodiment of the invention, an OCT system is configured for the acquisition of retinal images with blood vessels and capillaries emphasized; such images are referred to as angiograms. In one embodiment of the invention, the acquired images are divided into smaller sub-images, herein known as “strips”, by automatically detecting and removing motion artefacts caused by microsaccadic motion. In one embodiment, these strips are registered to a template image and averaged to form a single enhanced image. A template image is an image in which there are no detectable microsaccadic motion artefacts present. In another embodiment, these strips are registered to each other and averaged to produce a single enhanced image. In one embodiment, the set of acquired images contain corresponding images for several retinal layers. For example, an image set may contain corresponding images of the deep and superficial capillary layers of the retina. In this embodiment, the algorithm may be applied to each set of retinal layer images in parallel.

One aspect of the invention describes several possible execution paths that vary based on the image set that the invention is applied to. Examples of the possible variations of the invention's execution path are categorized under one of two embodiments of the invention described in detail below, which are known herein as the “template embodiment” and the “template-less embodiment”. The embodiment that is chosen is automatically decided based on the presence of a template image, or lack thereof, in the image set that the invention is being applied to. If a template image is present, the template embodiment is used. If no template image is present, the template-less embodiment is used. Each of these two main embodiments vary in their intermediate steps, but both embodiments produce images as an output.

### BRIEF DESCRIPTION OF THE DRAWINGS

The following drawings will be used to assist in the description of the invention by way of example only, and without disclaimer of other embodiments.

FIG. 1A shows a schematic representation of an OCT system used for imaging the eye.

FIG. 1B shows a schematic representation of a generalized OCT system.

FIG. 1C shows a schematic representation of a multi-scale OCT system with adaptive optics used for imaging the eye.

FIG. 1D shows a schematic representation of a multi-scale OCT system with adaptive optics used for imaging the eye. In this embodiment of the invention, the polarization properties of light are used to integrate a deformable optical element to control the wavefront incident on the eye.

FIG. 1E shows a schematic representation of a multi-scale OCT system with adaptive optics used for imaging the eye. In this embodiment of the invention, the deformable optical elements are illustrated as transmission optical elements.

FIG. 2 shows a schematic representation of an interferometric configuration for phase stabilization of the OCT.

FIG. 3A shows 1D scanning profile comparison between unidirectional and bidirectional scannings.

FIG. 3B shows 2D scanning profile comparison between bidirectional and step bidirectional scannings.

FIG. 3C shows 2D scanning profile of time interval increased bidirectional scanning.

FIG. 4 is a high-level flow chart of the registration and averaging process for OCT images.

FIG. 5 is an illustration of the generalized method for the invention, showing the target image (which may be a template image or a strip, depending on which embodiment of the invention is used), the images to be registered, the motion-free (e.g. microsaccade-free) strips, and the final output image.

FIG. 6 is an illustration of the results from the template embodiment, showing the template image as well as the mean images that are produced as output.

### DETAILED DESCRIPTION

This invention describes a novel systems and methods for the acquisition of OCT-A images.

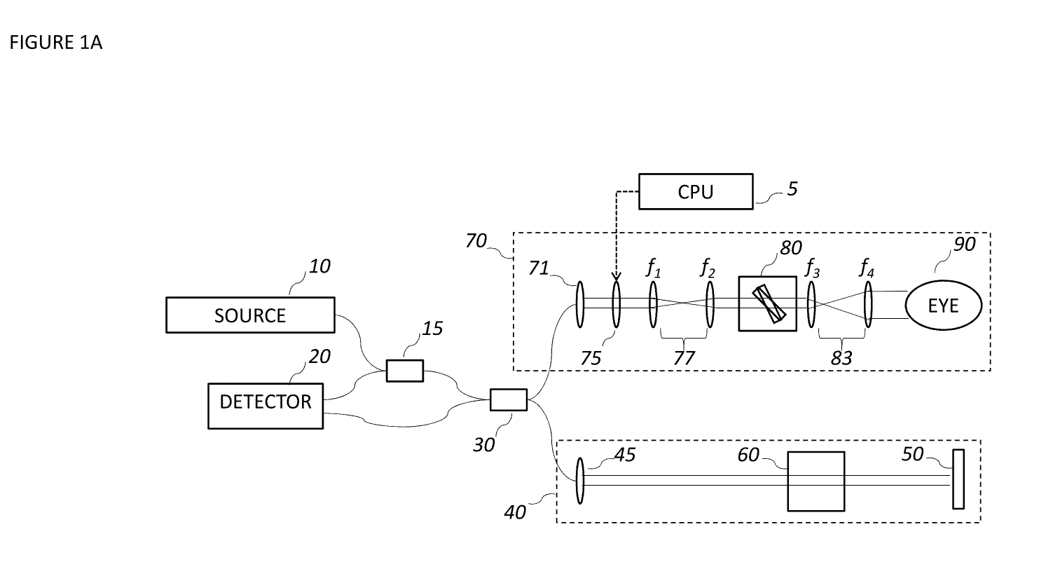

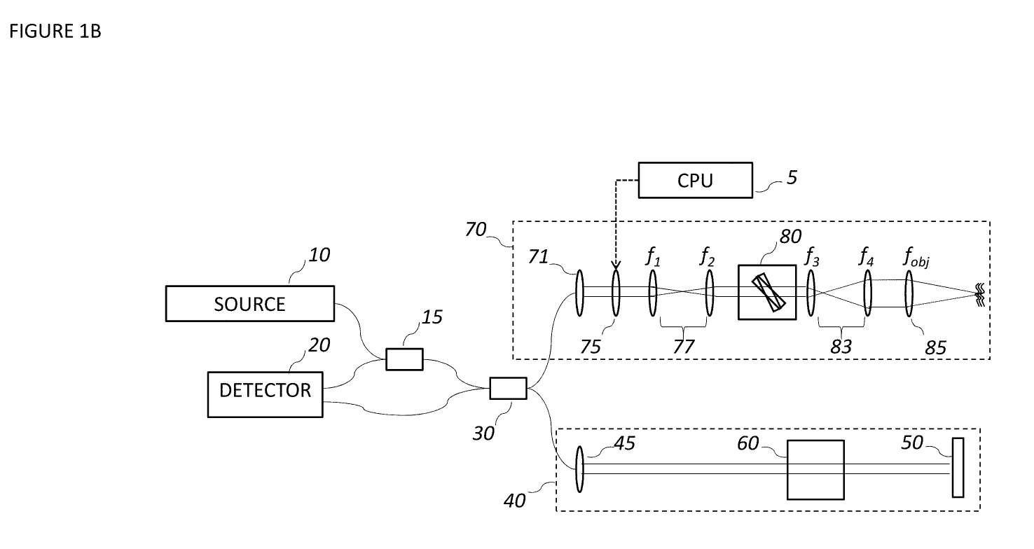

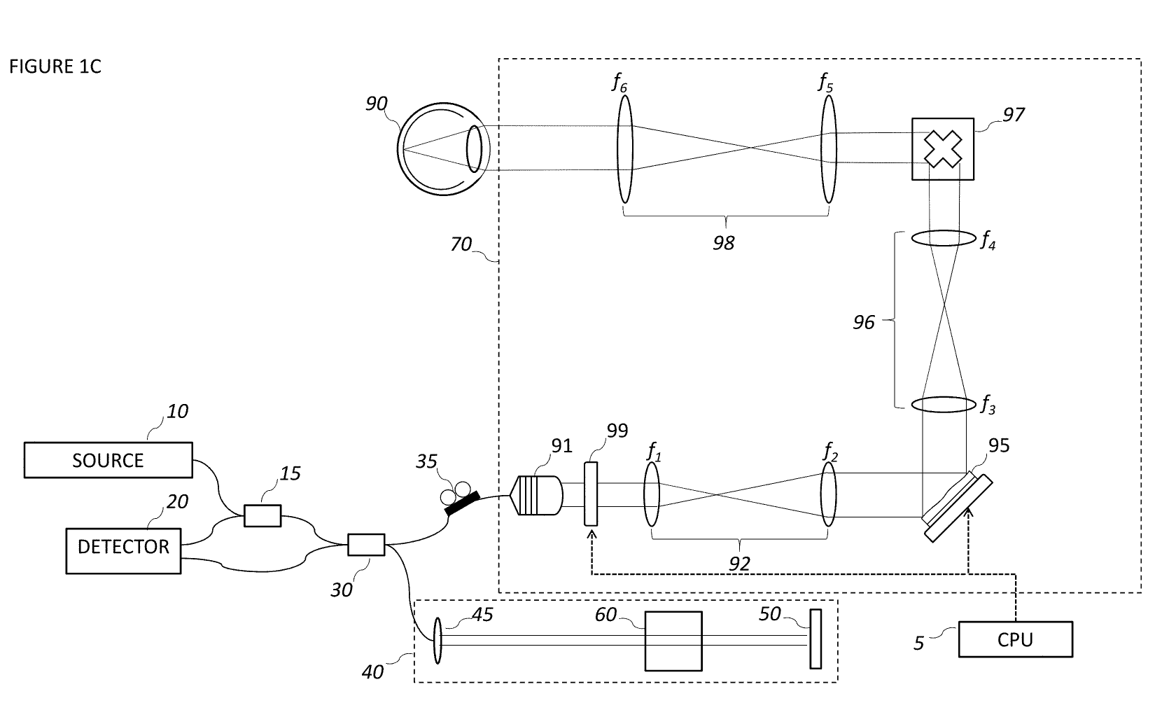

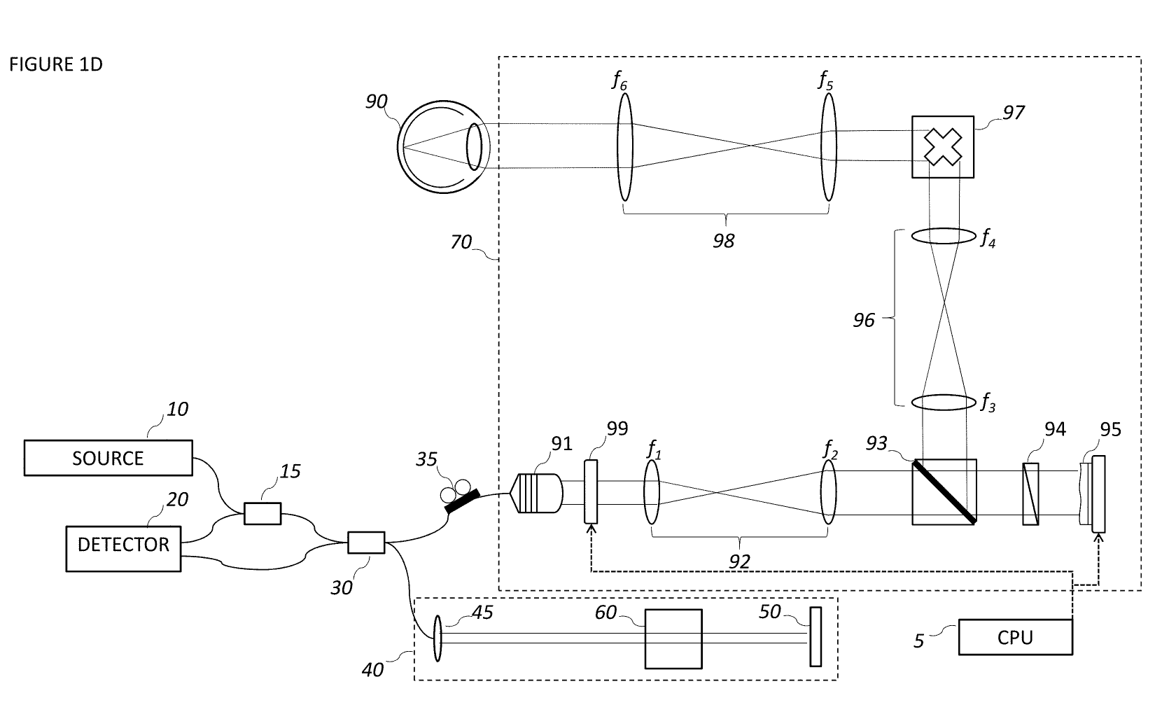

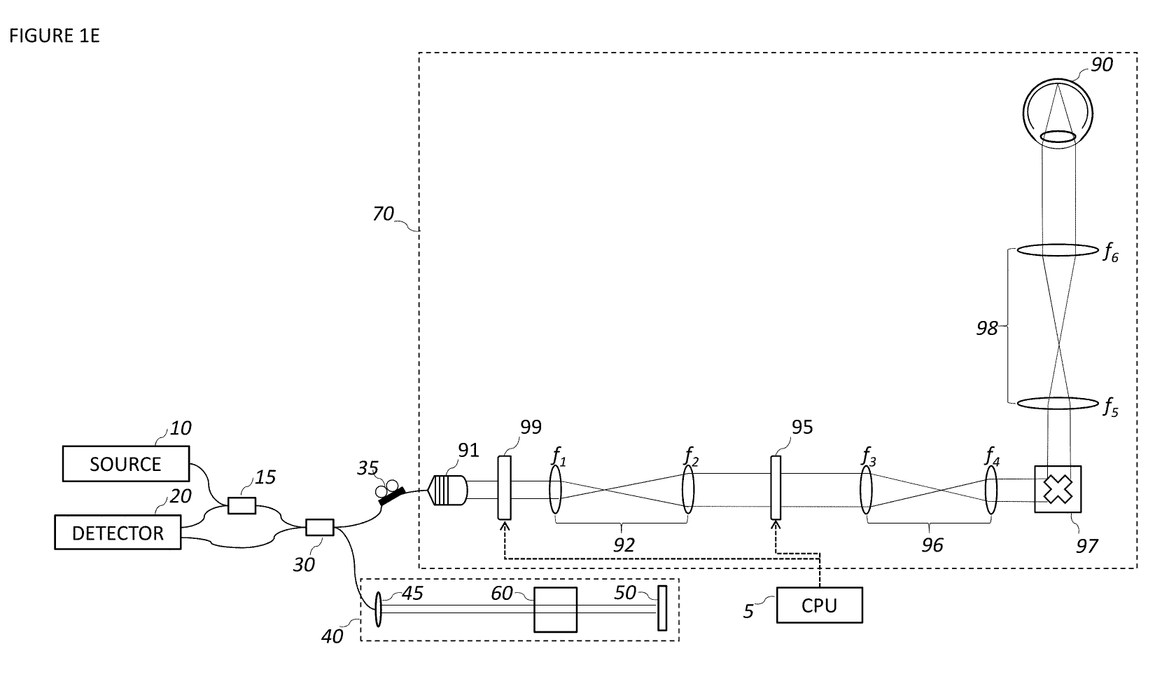

Demonstration of the invention were performed on custom developed prototype OCT or OCT-A acquisition systems. Examples of representative OCT embodiments are presented in FIG. 1A and FIG. 1B. In the demonstrated configuration, the OCT engine is based on a wavelength swept laser (or light source) (10), in a configuration known as Swept Source (SS) OCT or alternatively as Optical Frequency Domain Imaging (OFDI). The detector is a balanced photodiode (20). An unbalanced photodiode could also be used. The OCT system is computer controlled, combining and providing signals for timing and control, and for processing the interferometric data into images or volumetric data. It could be controlled using an embedded controller as well. The fibre coupler (30) splits the light from the source into reference (40) and sample (70) paths or arms. The fibre coupler may have a splitting ratio of 50/50, or some other ratio. In some embodiments, the fibre coupler (or fibre optic beam splitter) is replaced by a free-space beam splitter. The reference arm has a mirror (50) typically mounted on a translation stage and may contain dispersion compensating optical elements (60). In an alternate embodiment, the reference arm could be a fibre with a fibre-integrated mirror. In one embodiment, shown in FIG. 1A, the sample arm optics (70) are designed for imaging a retina, and the final objective is the cornea and intraocular lens of the eye (90). Light from the fibre is collimated by lens (71). A controllable wavefront modifying optical element (75), such as a variable focus lens controlled by a computer or processor (5) may be placed after the collimator. The light is passed through an optical relay (77) to a scanning mechanism (80) that is used to scan the angle of light incident on the cornea, which in turn scans the lateral position of the focused spot on the retina. An optical relay comprises of a 4-f configuration of lenses or curved mirrors. The elements of the optical relay are separated by the sum of their focal lengths. The optical relay optically conjugates the input focal plane to the output focal plane. Another optical relay (83) is used between the scanning mechanism (80) and the eye (90). The light returning from the sample and reference arms are combined through the beam splitter to create an interference signal and directed towards the detector (20). The optical interference signal is processed to construct a depth resolved image or images of the sample. The control of the scanning mechanism in the sample arm can be used to acquire three dimensional (3D) volumetric data of the sample. In some embodiments, the sample could be a human or an animal eye.

In another embodiment of the invention shown in FIG. 1B, the OCT system could be used to image another type of sample, in which case an objective lens would be used before the sample in the optical setup (85). In another embodiment, a controllable wavefront modifying optical element (75), such as a variable focus lens controlled by a computer or processor (5) may be placed after the collimator. The light is passed through an optical relay (77) to a scanning mechanism (80). Another optical relay (83) is used between the scanning mechanism (80) and the objective (85).

Alternative variations of this configuration could replace the SS OCT with a Spectral Domain/Spectrometer Domain (SD) OCT or a Time Domain (TD) OCT. For Spectral Domain OCT, the swept laser is replaced by a broad band light source, and the detector is spectrally resolved, for example, using a spectrometer. For Time Domain OCT, the swept laser is replaced by a broad band light source, and the reference mirror position is scanned axially (or angularly) to generate interference fringes. Thus, TD-OCT comprises of a scanning reference mirror; wherein the reference mirror is scanned to modulate an optical path-length in the reference arm. Operating wavelengths for retinal imaging are from the visible to near infrared. In one embodiment, the central wavelength is 1060 nm, with a bandwidth of ˜70 nm. In another embodiment, the central wavelength is 840 nm, with a bandwidth of ˜50 nm. Other embodiments may use combinations of central wavelength ranging from 400 nm to 1300 nm, and bandwidth of approximately 5 nm up to over 100 nm, and in some cases with central wavelengths around 700 nm and bandwidths of several 100 's of nanometers. In other embodiments, higher or lower source wavelengths could be used. In some embodiments the fibre coupler (15) is an optical circulator. In other embodiments of the system, the detection may not be balanced, and fibre coupler (15) may be replaced by a direct optical path from the source to the interferometer fibre coupler (30). Alternative variations of the interferometer configuration could be used without changing the imaging function of the OCT engine.

The instrument control sub-system (or instrument controller) may further comprise of at least one processor configured to provide timing and control signals; at least one processor for converting the optical interference to the 1 or 2 or 3-dimensional data sets. The processor(s) may extract a specific depth layer image from the three dimensional data sets, or a range of depth layer images. In one embodiment, the depth layers are summed along the axial direction to generate a depth projected image called a two-dimensional (2D) en face image. Other approaches of combining the axial depth information in the range of depth layers include maximum intensity projection, median intensity projection, average intensity projection, and related methods. A 2D depth layer extracted from a 3-D volume is called an en face image, or alternatively in some embodiments, a C-scan.

At each scan position, corresponding to a point on the specimen, the OCT signal acquired at the detector is generated from the interference of light returning from the sample and the reference arms.

In some embodiments, a common path OCT system may be used; in this configuration, a reference reflection is incorporated into the sample arm. An independent reference arm is not needed in this embodiment. Light from the source is directed into a 3 or 4 port beam splitter, which in some embodiments is a bulk optic beam splitter or a 2×1 fiber splitter, or which in other embodiments is an optical circulator. Light in the source arm is directed by the beam splitter to the sample arm of the common path interferometer. A reference reflecting surface is incorporated into the sample arm optics, at a location that is within the interference range of the sample. In some embodiments, the reference reflection may be located approximately integer multiples of the light source cavity length away from the sample, utilizing “coherence revival” to generate interference. Light returning from the sample and light returning from the reference reflecting surface are directed back through the beam splitting element toward the detector arm, generating interference fringes as with conventional OCT systems. The interference is digitized, and the remaining OCT processing steps are similar as with conventional interferometric configurations. By having a reference reflection integrated with the sample, common path interferometry is immune to phase fluctuations that may be problematic in conventional interferometers with two, or more, arms. In the case of using an optical circulator in the place of a 2×1 coupler, the efficiency is higher. The source and detector combination may comprise a spectral domain OCT system, or a swept source OCT system.

The interference signal is processed to generate a depth profile of the sample at that position on the specimen, called an A-scan. A cross-sectional B-scan image is generated by controlling the scanning of the position of the beam laterally across the specimen and acquiring a plurality of A-scans; hence a two dimensional (2D) B-scan depicts the axial depth of the sample along one dimension, and the lateral position on the sample in the other dimension. A plurality of B-scans collected at the same location is referred to as a BM-scan; the repeat acquisition of the B-scans at the same location is used to enhance motion contrast in the sample. The B-scan elements of particular BM-scan are denoted OCT, where i goes from 1 to the number of B-scans in the BM-scan. A collection of BM-scans acquired at different locations on the retina constitute a volume. The BM-scans are enumerated BMk, where k goes from 1 to the number of BM-scans in the volume. The i^{th} OCT B-scan of the k^{th} BM-scan in a volume can be indexed as BM-scan_{k,i}. In one embodiment, the BM-scans are acquired at different lateral positions on the sample in a raster-scan fashion. In another embodiment, the BM-scans are acquired in a radially spoked pattern. In other embodiments, the BM-scans are acquired in a spiral, or Lissajous, or other patterns, in order to acquire a volumetric image of the sample.

In another embodiment, the OCT system may be “full field”, which does not scan a focused beam of light across the sample, but rather uses a multi-element detector (or a 1-dimensional or 2 dimensional detector array) in order to acquire all lateral positions simultaneously.

In another embodiment, the OCT interferometer may be implemented in free space. In a free space interferometer, a conventional bulk optic beam splitter (instead of the fiber-optic splitter (30)) is used to divide the light from the source into sample and reference arms. The light emerging from the bulk optic beam splitter travels in free space (e.g., air), and is not confined in a fiber optic, or other form of waveguide. The light returning from the sample and reference arms are recombined at the bulk optic beam splitter (working as a beam combiner in the reverse direction) and directed toward at least one detector. The interference between the light returning from the sample arm, and the light returning from the reference arm is recorded by the detector and the remaining OCT processing steps are the same as with fibre based interferometric configurations. The source and detector combination may comprise a spectral domain OCT system, or a swept source OCT system, or a time domain OCT system.

Multi-Scale Oct with Adaptive Optics for High Lateral Resolution

In an embodiment of the invention, the optics used for light delivery to the sample may be configured for multi-scale imaging, meaning that the lateral resolution can be varied from low to high, and that adaptive optics may be used for the high lateral resolution mode. When adaptive optics are used for high resolution OCT imaging, the system is referred to as AO OCT.

Lateral resolution is increased by using a large diameter probe beam incident on the cornea. Standard “low resolution” retinal imaging conventionally uses a beam diameter incident on the cornea of approximately 1 to 1.5 mm. In one embodiment for high lateral resolution imaging, the beam diameter may be greater than 2.5 mm. In another embodiment, the increased lateral resolution is accompanied by adaptive optics in order to achieve a diffraction limited, or close to diffraction limited, focal spot at the retina. In one embodiment, the adaptive optics will comprise an optical element to shape the wavefront of the incident beam of light, such as a deformable mirror, deformable lens, liquid crystal, digital micromirror display, or other spatial light modulator. In one configuration, the shape of the controllable wavefront modifying optical element is determined using a wavefront sensor.

In another embodiment of the invention, a sensorless adaptive optics method and system may be used, in which a merit function is calculated on the image quality in order to control the wavefront shaping optical element. More detailed information on sensorless adaptive optics may be found at the following reference: Y. Jian, S. Lee, M. J. Ju, M. Heisler, W. Ding, R. J. Zawadzki, S. Bonora, M. V. Sarunic, “Lens-based wavefront sensorless adaptive optics swept source OCT,” Scientific Reports 6, 27620 (2016) and described in patent application PCT/US 2016/051369 titled “Coherence-Gated Wavefront-Sensorless Adaptive-Optics Multi-Photon Microscopy” filed September 12, 2016. The entire disclosure of the PCT/US 2016/051369 is hereby incorporated by this reference in its entirety for all of its teachings.

FIG. 1C is a schematic view of one embodiment of the invention with the light delivery optics to the sample configured for multi-scale imaging, having the capability of switching between high lateral (“high”) resolution and low lateral (“low”) resolution imaging modes. The Numerical Aperture (NA) of the imaging system is a metric of the lateral resolution, and is calculated based on the final diameter of the beam emerging from the sample arm (70) after the final relay (98) and the focal length of the sample, in this example the focal length of the refractive elements of an eye (90). Multi-scale imaging with different NA can be adjusted by controlling the diameter of the beam using the zoom collimator (91). In one embodiment, the zoom collimator (91) is continuously variable, and the output beam diameter after the zoom collimator (91) can be controlled between ˜1.3 mm and ˜3.9 mm. The magnifications of the optical relays (92, 96, and/or 98) can be used to determine a fixed system magnification from the zoom collimator (91) to the sample (90). In one embodiment, the combined magnification of the optical relays was ˜1.3 x, and the range of beam diameter settings delivered to the cornea of the eye (90) was at low NA equal to 1.7 mm and at high NA equal to 5.0 mm. Since the zoom collimator (91) was continuously adjustable, any beam diameter between the minimum NA and maximum NA could be selected. Assuming a 22.2 mm focal length of the eye and refractive index of 1.33 for a water at 1.06 μm, the range of transverse resolutions for multi-scale imaging was nominally estimated to be 10.6 μm and 3.6 μm with the low and high NAs respectively, as defined by using the equation for transverse resolution Δx=0.51 λ/NA. In one embodiment, an additional deformable optical element, such as a variable focal length lens, or a deformable mirror, or a liquid crystal spatial light modulator, could be placed at location (99). Element (99) is illustrated as a transmissive optical element, in another embodiment it may be a reflective optical element.

Another embodiment of the invention is presented in FIG. 1D using the polarization properties of light to incorporate a controlable optical element (95) into the sample arm to control the wavefront incident on the eye. The probe beam from a zoomable collimator (91) directs the light through an optical relay (92) to a polarizing beam splitter (PBS, 93), and the P-polarization beam passes through the PBS while the S-polarization light is reflected and rejected from the system. After passing through the PBS, the polarization plane of the P-polarization is rotated by 90° using an achromatic zero-order quarter wave plate (QWP, 94) oriented at 45°, reflected by the deformable optical element (95) and reverse passes through the QWP (94). The deformable optical element (95) may be a deformable mirror (DM), or a deformable transmissive lens followed by a mirror, or a liquid crystal spatial light modulator followed by a mirror. After the second pass through the QWP (94), the polarization of the beam is rotated 90°, becoming an S-polarization beam, and is reflected by the PBS (93) and directed through an optical relay (96) to the scanning mechanism (97), which provide scanning of the beam at the sample. Following the scanning mechanism, the light passes through an optical relay (98) and is directed to the sample, in this case an eye (90). In one embodiment, an additional deformable optical element, such as a variable focal length lens, or a deformable mirror, or a liquid crystal spatial light modulator, could be placed at location (99). Element (99) is illustrated as a transmissive optical element, in another embodiment it may be a reflective optical element. The combination of (99) and (95) may be used as a woofer-tweeter configuration, where one deformable optical element is used to correct low order aberrations, and the other is used to correct for high order aberrations.

In the embodiment of the invention presented in FIG. 1D, polarization optics are used to make a compact adaptive optics configuration. In the above specific example only the P-polarization of light at the zoom collimator (91) was used for OCT imaging. By adjusting a polarization controller (35) before the zoom collimator (91), the optical powers of the two orthogonal polarization states are controlled to maximize the light in the P-polarization. In another embodiment, the S-polarization could be used for imaging.

In another embodiment of the invention, additional polarization controlling optics maybe be inserted in the beam path to change the polarization of the light illuminating the sample, for example, to rotate the polarization of the linearly polarized light 45° or 90°, or to generate light that is circularly polarized. Acquisition of OCT images of the sample with different polarizations may be used to generate polarization contrast.

Multi-scale imaging with a zoom collimator (91) may be used with adaptive optics configurations using deformable lenses for aberration corrected imaging. An embodiment of the invention is presented in FIG. 1E, with the deformable optical element (95) illustrated as a transmissive deformable lens. In one embodiment, an additional deformable optical element, such as a variable focal length lens, or a deformable mirror, or a liquid crystal spatial light modulator, could be placed at location (99). Element (99) is illustrated as a transmissive optical element, in another embodiment it may be a reflective optical element. The combination of (99) and (95) may be used as a woofer-tweeter configuration.

In other embodiments of the system, the zoom collimator may be replaced by a zoom relay, for example at the position of optical relay (92) to provide multi-scale imaging.

### Phase Stablization for Oct

The wavelength-scanning light source is used for swept source (SS) OCT scanned by changing the wavelength along the temporal axis. Phase stability is affected by jitter between the scanning of the wavelength of the light source and the timing at which data is acquired by the optical detector. This fluctuation of the signal waveforms in the time-axis affects the spectral interference signals, resulting in image distortion. The amount of temporal phase jitter or spectral shift that originates in the source is denoted as β.

When on a small scale, this jitter introduces random phase shifting to the spectral sampling (in time) and consequently causes jitter of the spectral interference signals obtained by the SS OCT.

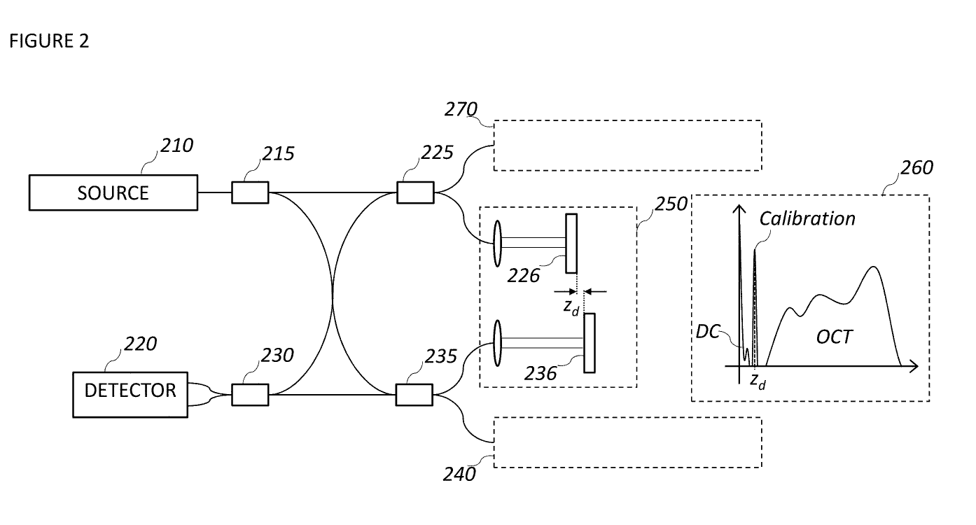

In one embodiment of the present invention, phase stabilization is made possible by employing an additional interferometer to generate a calibration signal. One embodiment of a phase stabilized interferometer configuration is presented schematically in FIG. 2.

The calibration signals are generated as described below using the interferometric signal generated by the phase stabilization unit (250). In the phase stabilization unit (250), the mirrors in the sample and reference arms of the calibration interferometer (226) and (236) are placed with a differential optical path length z_{d} with respect to each other. The two beams reflected from the mirrors travel through the couplers (225) and (235), and generate a fixed interference signal at the coupler (230). This interference signal is detected by the detector (220), and functions as the phase stabilization calibration signal. The overall optical path of the calibration signal, from the couplers (225) and (235) to the reflecting mirrors (226) and (236) is made to be either significantly longer or shorter than overall optical path of the OCT interferometer, on the order of, for example 2 m, so that it does not interfere with the OCT signals.

In order to avoid overlapping between the calibration interferometer signal and the OCT signals, the depth position of the calibration signal corresponding to the z_{d} is set to be close to DC in spatial frequency domain (depth) as presented in Diagram of FFT signal (260). In another embodiment, z_{d} may be set deeper than the anticipated features to be imaged.

There are multiple methods of generating a calibration signal. In other embodiments, the calibration signal can be generated by a Mach Zehnder interfometer, or a common path interferometer. In other embodiments, the calibration signal could be generated using an etalon (e.g. Fabry-Perot interferometer) in bulk optics or in fiber. In other embodiments, a calibration signal can be generated by using an autocorrelation signal inside of the reference arm of the OCT interferometer, for example using a thin piece of glass with parallel sides, such as a microscope coverslip. In other embodiments, a calibration signal could be integrated with the OCT interferometer sample and reference arms.

There are multiple ways of calculating the amount of phase jitter or spectral shift arising from the source spectrum. One embodiment of the invention for phase jitter compensation is explained below. The calibration signal from the first A-line of a B-scan is used as the reference and its detected spectral intensity is given by Iᵣ(j). The target calibration signal from the other A-line data in the B-scan to be re-calibrated is given by Iₜ(j). The relationship of these two calibration signals is represented by the formula (Mathematical Formula 1) below,

Iᵣ(j)=|E_{A}(j)+E_{B}(j)|²

Iₜ(j)=|E_{A}(j−β)+E_{B}(j−β)|²=Iᵣ(j)*δ(j−β) [Mathematical Formula 1]

where E_{A}(j) and E_{B}(j) are the sampled spectra of the two beams reflected from the mirrors (226) and (236) in the phase stabilization unit (230) with sampling index of j. Here, * represents convolution, while β indicates a relative phase jitter or spectral shift that originates in the source and is measured in the A-scans with respect to the reference calibration signal, Iᵣ.

For the spectral shift estimation, the complex conjugates of the Fourier-transformed reference and target calibration signals are integrated according to the formula (Mathematical Formula 2) below,

$$[ Mathematical Formula 2 ]$$

$$\begin{matrix}ℱ [ {I}_{r} ( j ) ] {ℱ [ {I}_{t} ( j ) ]}^{*} = ℱ [ {I}_{r} ( j ) ] ℱ [ {{I}_{r} ( - j )}^{*} ] ℱ [ δ ( - j - β ) ] \\ = ( ξ ) ( ξ ) {e}^{( \frac{- i 2 πξβ}{N} )}\end{matrix}$$

where [symbol] [·] represents Fourier transformation, ξ is the index of the Fourier transformation of j, N is the number of sampling points, superscript * indicates complex conjugation, and the accent ‘˜’ represents the Fourier transformed signal.

Unless two calibration signals (reference and target) are fully correlated, the phase difference between the two calibration signals is expressed by the formula (Mathematical Formula 3) below,

Δψ(ξ)=Arg[ [symbol] (ξ) [symbol] (ξ)]=^{-2πξβ}/_{N} [Mathematical Formula3]

where Arg [X] is a function that gives the phase components of the complex number X. This is also referred to as the “angle” of the complex number when expressed in polar form.

In swept-source based OCT, the light source is scanned in time with respect to the wavelength of light. When processing the OCT data, acquired raw OCT data must be converted to become linear along the wavenumber (k), which is done through a procedure called re-scaling that involves table conversion. The interferogram needs to be rescaled to be linear in wavenumber prior to Fourier transformation into spatial coordinates. This can be done using a look up table (LUT).

Because the spectral shift estimation is performed before the re-scaling process, the phase difference slope, Δψ(ξ), cannot be directly used for the spectral shift compensation.

For the spectral shift compensation, the phase difference between the two calibration signals calculated at the position of the peak, Δψₚ=Δψ(ξₚₑₐₖ) is extracted and multiplied to the Fourier transformed OCT signal after the re-scaling process as described by the formula (Mathematical Formula 4) below,

$$ℱ [ {I}_{OCT} ( j ) ] = ℱ [ {I}_{OCT} ( {LUT}_{j \to k} ( j ) ) ] = {\tilde{I}}_{OCT} ( z ) {e}^{- i \frac{{Δϕ}_{p}}{{z}_{d}} z} \qquad [ Mathematical Formula 4 ]$$

where I_{OCT}(j) is the spectral intensity of OCT signal, LUT is the look-up-table for re-scaling the interferogram to be linearly sampled in wavenumber, z is the index of the Fourier transformation of k (also referred to as depth in OCT image) and z_{d}is the depth position of the calibration signal peak. Because the phase difference caused by the spectral shift is linear in wavenumber, the linear fit with the slope (Δψₚ/z_{d}), can be directly applied to the wavenumber rescaled OCT signal.
Visualization of the Microvasculature with Oct Angiography

In some embodiments, the back-scattering intensity contrast of the blood vessels relative to the surrounding tissue may provide adequate contrast for visualization of the retinal blood vessels. In one embodiment, the increased contrast from the blood vessels may arise due to high resolution retinal imaging.

In one embodiment, the parameters of the OCT acquisition system and the parameters of the processing methods are used to enhance the contrast of flowing material; this system and method is referred to as OCT-Angiography (OCT-A). In one embodiment, the features of interest in OCT-A are blood vessels or capillaries, which may be visualized in B-scans, or en face images, or in the 3D OCT volumetric datasets. OCT images with blood vessel or capillary contrast are referred to as angiograms. Flow contrast to enhance the appearance of the blood vessels in the angiograms may be performed using any number of methods.

Comparison of (or monitoring) the difference or variation of the OCT (or interferometric) signal (e.g. between B-scans in a BM-scan) on a pixel-wise basis enhances the contrast of the blood flow in the vessels relative to the static retinal tissue.

In one embodiment of flow contrast enhancement of blood vessels, called speckle variance (sv) OCT, the pixel-wise comparison is performed by calculating the variance of the intensity values at the corresponding pixels in each B-scan of a BM-scan. An example of the equation used to compute each speckle variance frame (svⱼₖ) from the intensity data from the BM-scans in an OCT volume (Iᵢⱼₖ) is

$${sv}_{jk} = \frac{1}{N} {\sum }_{i = 1}^{N} {( {I}_{ijk} - \frac{1}{N} {\sum }_{i = 1}^{N} {I}_{ijk} )}^{2} ,$$

where I is the intensity of a pixel at location i,j,k; i is the index of the B-scan frame; j and k are the width and axial indices of the i^{th} B-scan; and Nis the number of B-scans per BM-scan. In one embodiment, the volume size is j=1024 pixels per A-scan, k=300 A-scans per B-scan, and N=3 for a total of 900 B-scans (300 BM-scans) per volume.

In another embodiment of flow contrast enhancement called phase variance (pv) OCT, the calculation utilizes the phase of the OCT signal or the optical interference signal. In another embodiment, the calculation utilizes the complex OCT signal. In other embodiments, the number of B-scans per BM-scan may be as low as 2, or greater than 2. In other embodiments, the OCT A-scan in the spectral domain may be divided into multiple spectral bands prior to transformation into the spatial domain in order to generate multiple OCT volume images, each with lower axial resolution than the original, but with independent speckle characteristics relative to the images reconstructed from the other spectral bands; combination of the flow contrast signals from these spectral sub-volumes may be performed in order to further enhance the flow contrast relative to background noise. Other embodiments of flow contrast to enhance the appearance of blood vessels may include optical microangiography or split spectrum amplitude decorrelation angiography, which are variations of the above mentioned techniques for flow contrast.

### Bidirectional Oct Angiography

Various scanning devices such as dual-axis galvanometer-based scanners (GS), a combination of a single GS and a polygon mirror, and microelectromechanical based resonant scanners, are utilized for high speed 3D volumetric OCT image acquisition. Among them, dual-axis GS are the most commonly utilized devices for later scanning in OCT system due to several advantages over the other scanning devices, including: precision positioning, adjustability of scanning frequency, larger scanning angle, and compact constructive solution at a reasonable cost. Lateral scanning with dual-axis GS commonly proceeds in a raster fashion, so that one dimension (fast axis) is scanned rapidly while another dimension (slow axis) is scanned slowly. The most widely utilized GS driving functions are sawtooth, sinusoidal, cycloidal, and triangular functions.

In one embodiment of the invention, a dual-axis GS bidirectional scan is employed for acquisition of OCT-A or SAO OCT-A data. The bidirectional scan is implemented by driving the fast axis with a triangular function and sampling on both the forward and return scan directions.

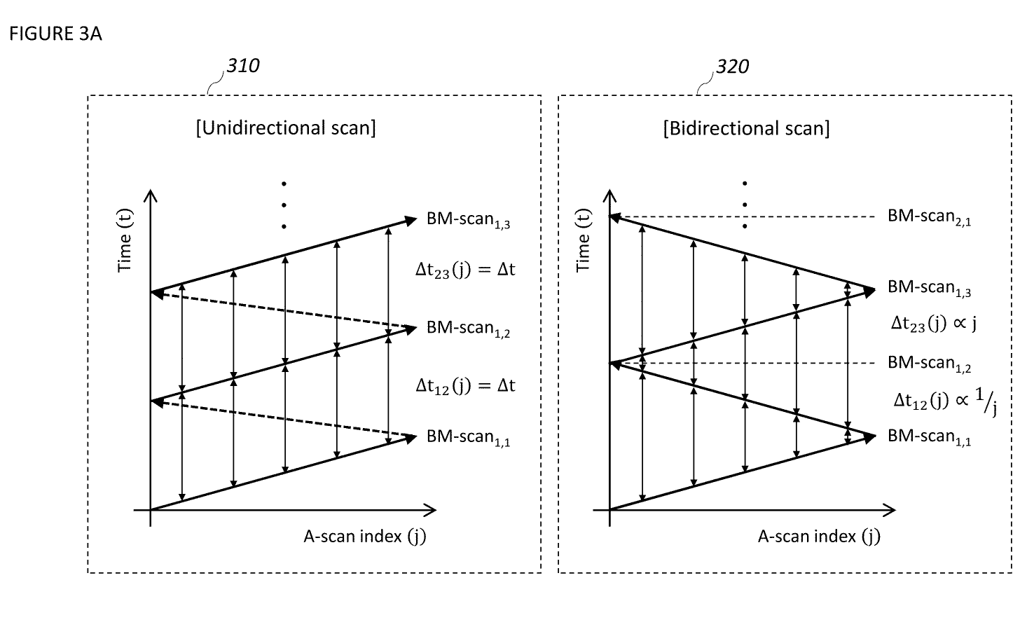

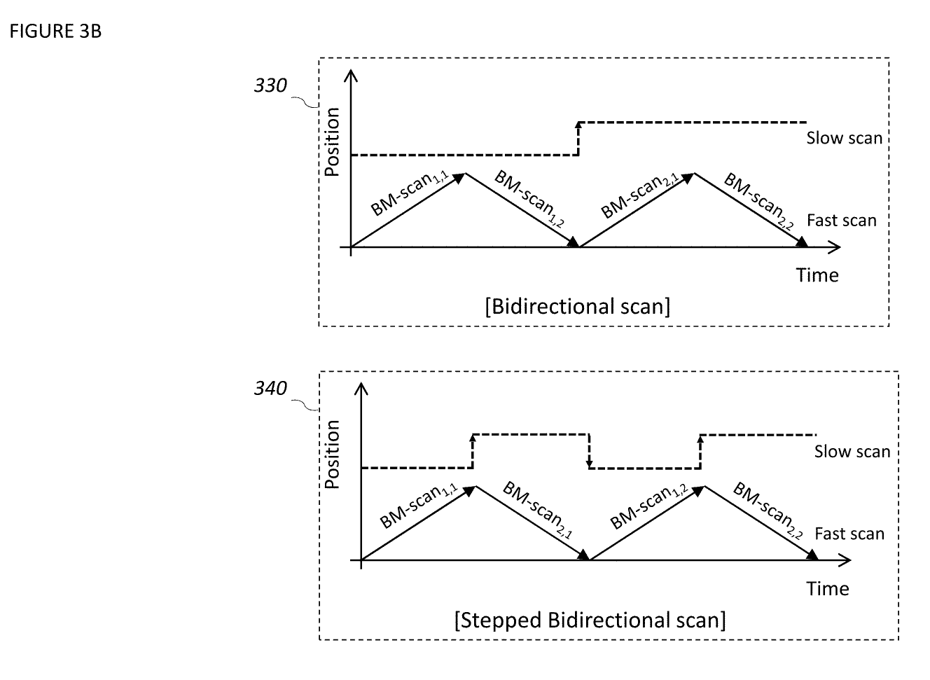

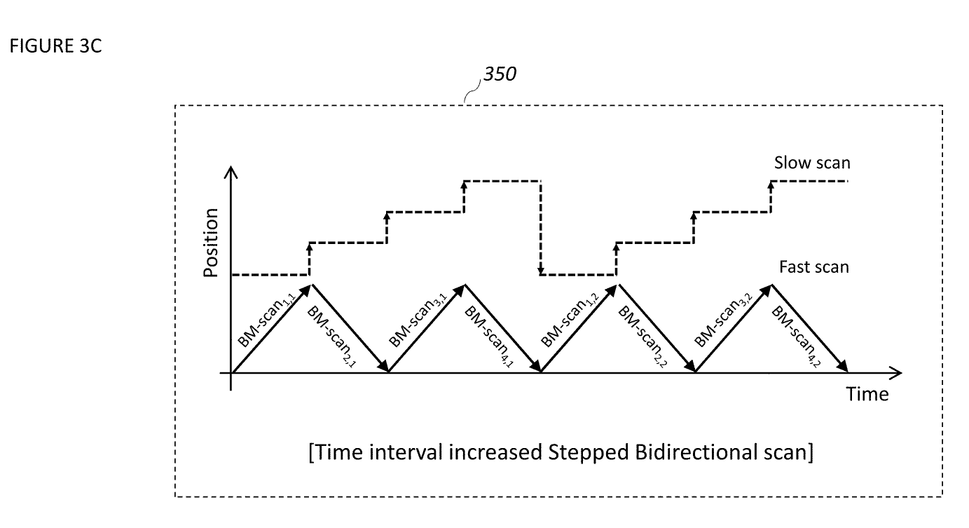

The differences unidirectional and bidirectional scanning protocols are illustratively presented in FIG. 3A. In the case of the unidirectional scan (310), a flyback scan (marked by the dashed arrow) is required to place the scanner back to the initial scan position. Useful image data is not collected during the flyback scan, and the time required for the flyback reduces the duty cycle of the scanning protocol. Hence, the speed of the flyback scan should be as fast as possible with the GS, without damaging them, introducing oscillations, etc. On the other hand, in the case of a bidirectional scan (320), at the end of a forward scan, a backward scan is performed with the same parameters as the forward scan, and data is sampled in both the forward and backward directions. Due to an absence of the flyback, a high scanning duty cycle can be achieved with the bidirectional scan.

Another unique aspect of the bidirectional scan is a time interval variation of A-scans between adjacent B-scans. As shown in FIG. 3A for the case of the unidirectional scan (310), the time interval (Δt) between corresponding A-scans in successive B-scans is constant. On the other hand, in the case of the bidirectional scan (320), the time interval between corresponding A-scans in sequential forward and backward B-scans at the same location is not a constant, but changing along the A-scan index. Depending on the B-scan index, the time interval is approximately linearly increasing for corresponding A-scans between B-scans acquired in the backward scan direction followed by forward scan direction, or linearly decreasing for corresponding A-scans between B-scans acquired in the forward scan direction followed by the backward scan direction. The OCT-A signal strength is related to the amount of time elapsed between corresponding A-scans and the speed of the flow in the sample. Hence, the time interval variation between corresponding pixels in a BM-scan comprising forward and backward scans at the same location introduces OCT-A image contrast variation along the scanning direction.

In one embodiment of the invention, OCT-A or SAO OCT-A is performed with the bidirectional scan using a BM-scan protocol comprising of three sequentially acquired B-scans. The bidirectional scan acquires data on the backward scan direction to improve the image quality of the angiography. We define a Time-variant Differential Complex Variance (TvDCV) algorithm for high contrast angiogram formation from the bidirectionally scanned three BM-scan OCT data set. The TvDCV algorithm is explained below. The three bidirectional BM-scanned complex OCT signals (Ĩ_{OCT}_{ᵢ}) at a given location and time are expressed by the formula (Mathematical Formula 5) below,

Ĩ_{OCT}_{₁}(x, z; t₁)=A_{OCT}_{₁}(x, z; t₁)e^{iφ}^{_{OCT 1}} ^{(x,z;t}^{₁}⁾

Ĩ_{OCT}_{₂}(x, z; t₂)=A_{OCT}_{₂}(x, z; t₂)e^{iφ}^{_{OCT 2}} ^{(x,z;t}^{₂}⁾

Ĩ_{OCT}_{₃}(x, z; t₃)=A_{OCT}_{₃}(x, z; t₃)e^{iφ}^{_{OCT 3}} ^{(x,z;t}^{₃}⁾ [Mathematical Formula5]

where A_{OCT}_{ᵢ} and φ_{OCT}_{ᵢ} correspond to the OCT signal amplitude and phase, respectively. Here, because of the bidirectional scan property shown in FIG. 3A, the even numbered index B-scans (backward scans) must be flipped horizontally to match the orientation of the forward scans.

From the three BM-scanned complex OCT signals, a set of three complex differential signals are obtained by the formula (Mathematical Formula 6) below,

ΔĨ_{∓}(x, z; Δt_{∓})=Ĩ_{OCT}_{₂}(x, z; t₂)e^{-iφ}^{₁₂}^{^{Bulk}}^{(x;Δt}^{₁₂}⁾−Ĩ_{OCT}_{₁}(x, z; t₁)

ΔĨ_{±}(x, z; Δt_{±})=Ĩ_{OCT}_{₃}(x, z; t₃)e^{-iφ}^{₂₃}^{^{Bulk}}^{(x;Δt}^{₂₃}⁾−Ĩ_{OCT}_{₂}(x, z; t₂)

ΔĨ_{c}(x, z; Δt_{c})=Ĩ_{OCT}_{₁}(x, z; t₁)e^{-iφ}^{₃₁}^{^{Bulk}}^{(x;Δt}^{₃₁}⁾−Ĩ_{OCT}_{₃}(x, z; t₃) [Mathematical Formula6]

where each subscript (∓, ±, c) represents the time interval (increasing, decreasing, and constant) along the A-scan index. A part of the processing is to remove the bulk phase offset that arises from the axial motion of the sample (e.g. an eye) during the acquisition of the BM-scan. The bulk phase offset φᵢⱼ^{Bulk} is estimated by the formula (Mathematical Formula 7) below,

$$[ Mathematical Formula 7 ]$$

$${φ}_{ij}^{Bulk} ( x ; Δ {t}_{ij} ) = Arg [ {\sum }_{z} {\tilde{I}}_{{OCT}_{j}} ( x , z ; {t}_{j} ) {\tilde{I}}_{{OCT}_{i}}^{*} ( x , z ; {t}_{i} ) ]$$

where Σ_{z} represents a summation of all pixels along the depth.

The TvDCV OCT-A image is generated by performing the variance operation on the three complex differential signals using the formula (Mathematical Formula 8) below,

$$[ Mathematical Formula 8 ]$$

$$TvDCV ( x , z ) = \frac{{\vert Δ \tilde{I} ∓ ( x , z ; Δ t ∓ ) + Δ \tilde{I} \pm ( x , z ; Δ t \pm ) + Δ {\tilde{I}}_{c} ( x , z ; Δ {t}_{c} ) \vert}^{2}}{3} - {( \frac{Δ \tilde{I} ∓ ( x , z ; Δ t ∓ ) + Δ \tilde{I} \pm ( x , z ; Δ t \pm ) + Δ {\tilde{I}}_{c} ( x , z ; Δ {t}_{c} )}{3} )}^{2}$$

In general, increasing the number of BM-scans increases the angiogram contrast, but at the expense of more time required for the OCT-A data acquisition. Since OCT-A imaging, and especially SAO OCT-A imaging, is extremely motion-sensitive, decreasing acquisition time is critical to minimize the artifact induced by eye motion.

FIG. 3B presents two different bidirectional scanning profiles using two BM-scans. In the first case of the bidirectional scan (330), the slow scan amplitude is changed in increments of the step size after performing the forward and backward scans (two BM-scans). As described above (in paragraph [0059]), due to the opposite scanning direction between the two B-scans, the time interval between corresponding A-scans in the adjacent B-scans is not a constant. This variation in the time interval causes a variation in the OCT-A contrast along the scanning direction.

In another embodiment of the invention, a stepped bidirectional scanning protocol is developed for effective two BM-scan bidirectional OCT-A or SAO OCT-A imaging. In the case of the stepped bidirectional scan (340), the slow scan amplitude is modulated at the frequency of the fast scan with a step function. For the case of two BM-scans, the amplitude of the slow scan is stepped up, down, and up again during the acquisition of four fast scans. As a result, the four B-scans are acquired at two different elevations (the scan direction perpendicular to the fast axis). Because the scan direction is constant for a particular elevation, a constant time interval between the two BM-scans can be achieved.

For the step bidirectionally scanned two BM-scans OCT data, a complex differentiation (CD) based angiography algorithm is applied to generate OCT-A image.

The complex differentiation (also referred to as Optical microangiography (OMAG)) is processed by the formula (Mathematical Formula 9) below,

CD(x, z)=|Ĩ_{OCT}_{₁}(x,z)−Ĩ_{OCT}_{₂}(x, z)e^{iφ}^{_{bulk}}^{(x)}| [Mathematical Formula 9]

where φ_{bulk} is the bulk phase offset estimated by the formula (Mathematical Formula 10) below,

$$[ Mathematical Formula 10 ]$$

$${φ}_{bulk} ( x ) = Arg [ {\sum }_{z} {\tilde{I}}_{{OCT}_{2}} ( x , z ) {\tilde{I}}_{{OCT}_{1}}^{*} ( x , z ) ]$$

As increasing OCT acquisition speed, time interval between the two BM-scans is getting short. Since OCT-A contrast highly depends on the strength of signal decorrelation at the non-static tissue (e.q., vasculature), longer time interval between the two BM-scans is desired, especially for imaging small capillaries.

In another embodiment of the stepped bidirectional scan protocol, the number of steps for the slow scan direction can be greater than two. In FIG. 3C, a stepped bidirectional scanning profile (350) with increased time interval is presented. In this example of the stepped bidirectional scanning protocol (350), the slow scan is stepped up four times during the four B-scans, then stepped down to the initial position, and stepped up again four times again. During this one cycle of the two BM-scan, four different BM-scan sets at the four different elevations are acquired. The increased time interval stepped bidirectional scan enables a longer interval between the repeated scans of a two BM-scan OCT sequence without any changes in a total acquisition time and sampling density for a volume. Although the cases of 2 steps and 4 steps are presented as specific examples, any even integer number of steps may be used, for example, 2, 4, 6 etc., all the way up to the limit of the number of BM-scans in a volume.

### Strip-Based Registration and Averaging of Serial Acquisition

In one embodiment of the invention, the imaging device is an OCT system or an AO OCT system. In one embodiment, the processor of the OCT system generates images comprising flowing material. In another embodiment, the processor generates angiograms. In other embodiments, the retinal images may be acquired by a retinal fundus camera, Scanning Laser Ophthalmoscopy, Photo-Acoustic Microscopy, laser speckle imaging, retinal tomography (e.g., Heidelberg Retinal Tomography), retinal thickness analyzer, or other technique that provides adequate contrast of the blood vessels. With any of these techniques, contrast agents may be used to enhance the visibility of the vessels, for example, fluorescence retinal angiography using a retinal fundus camera. One may use contrast agents such Fluorescein or Indocyanine Green (ICG) to enhance the contrast of blood vessels.

Thus, the image generated could be an en face image, or an angiogram or an en face angiogram. In one embodiment, the angiograms are superimposed on intensity images.

In one embodiment, for the purpose of demonstration of the invention, ten OCT-A volumes are acquired using a graphics processing unit-accelerated OCT clinical prototype. The OCT system can use (as an example, not by limitation) 1060-nm swept source 1060-nm swept source with 100 kHz A-scan rate and a 111-nm 10-dB tuning bandwidth. The digitized spectrum used for imaging corresponds to an axial resolution of ˜6 μm in tissue. The size of the focal waist on the retina was estimated using the Gullstrand-LeGrand model of the human eye to be ω₀≈7.3 μm (calculated using Gaussian optics) corresponding to a lateral FWHM of ˜8.6 μm. The scan area can be sampled (as an example, not by limitation) in a 300×300 grid with a ˜2×2 mm field of view in 3.15 s. Without loss of generality, in other embodiments, the scan area may be in the range of <1 mm to>15 mm, the sampling dimensions may be in the range of 20 to 10,000, or larger, per scan dimension, or the number of B-scans per BM-scan may be between 2 and 10, or larger.

Following acquisition with an imaging device, the images are stored on an electronically readable medium. At least two of such images are acquired serially, and preferably more than two images, are stored in a data structure. In one embodiment, this data structure is an array, where each cell of the array contains an image. If an image without motion artifact is available, it is designated as the template, and strips from the other images are registered to it. In the absence of a motion free image, the template-less embodiment is selected to proceed. The template-less embodiment uses a large strip as the initial template, but with each iteration, the template image may be updated by region growing for the target strips that extend beyond the template of the previous iteration. In this template-less embodiment, the template is iteratively generated by mosaicking target strips. With either template or template-less embodiment, the remaining images are subjected to an image processing step whereby motion artefacts (e.g. introduced by microsaccadic motion) are removed by dividing the images into motion-free (or microsaccade-free) strips.

An overview of the method is as follows. After dividing the images into strips, the strips undergo a transformation using an initial (coarse) registration to the template image. If the images are averaged after only this coarse registration, the details in the images may be lost because of small motion artefacts (caused by slow motion, for example smooth tremor or drift). Hence, the course registration, which brings common features in the strip images into close proximity, is followed by a fine registration, using non-rigid registration by pixel-wise local neighborhood matching. The details of the fine registration are described below. Briefly, the fine registration is performed by selecting a pixel in the target strip and extracting a local neighborhood around that pixel in target strip. This patch of the target strip is compared with a small search range in the template. The location of the template that has the highest similarity to the patch determines the transformed location of the selected target pixel (at the center of the patch). This process is repeated for each pixel in the target strip. Upon completion of the fine registration process, the transformed strips are averaged to produce a final image. In one embodiment, the averaging process uses a mean calculation. In another embodiment, the averaging process uses a median calculation.

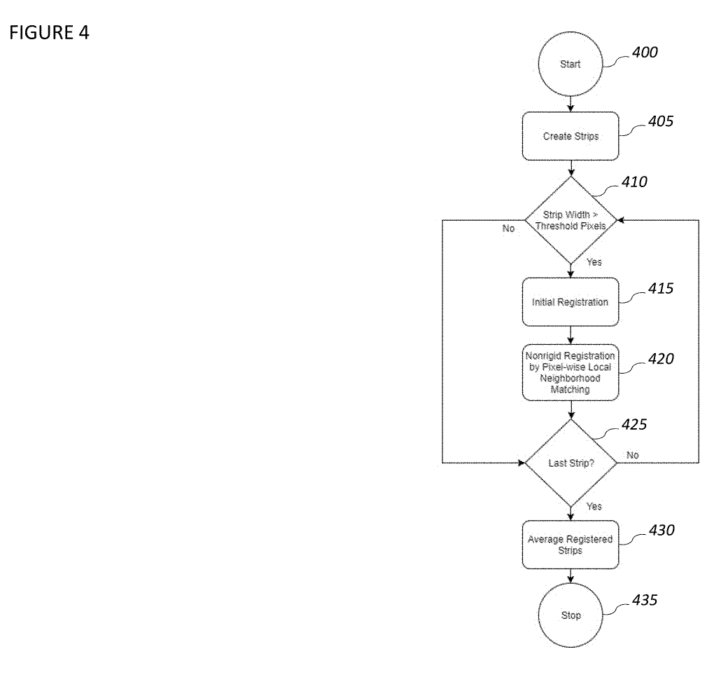

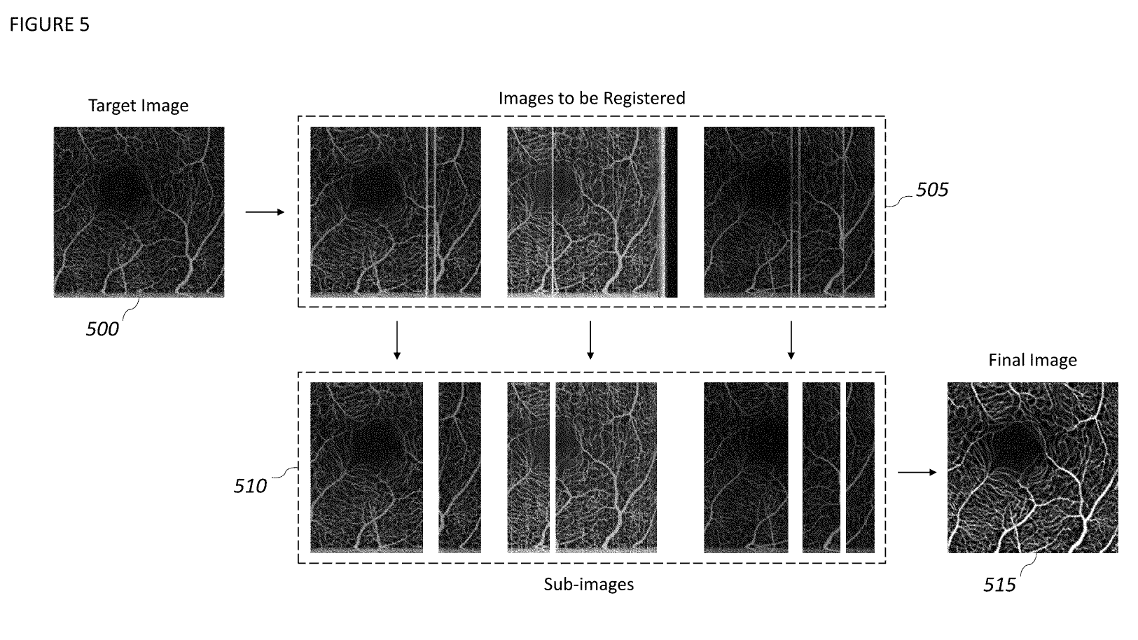

FIG. 4 represents a high-level flow chart for the motion corrected averaging and registration of OCT-A images. To start (400) OCT-A data is acquired with an OCT system, possible embodiments of which are shown in FIG. 1A, FIG. 1B and FIG. 1C. The acquired images are stored on an electronically readable medium, where one of these images may be a template image. If there is a template image, the template embodiment is chosen to proceed. If no template image is present in the image set, the template-less embodiment is chosen to proceed. The images are subjected to an image processing step whereby (e.g. microsaccadic) motion artefacts are removed automatically, creating free strips (405). This process is illustrated in FIG. 5. The strips are stored in a data structure. In one embodiment, the data structure is an array, where each cell of the array contains one strip. The first strip in the array is evaluated for size, and is discarded if its width is below a certain threshold number of pixels (410). In one embodiment of the invention represented by FIG. 4, the threshold is equal to 40 pixels. If the width of the strip is greater than the threshold, the strip undergoes an initial registration process (415). The purpose of the initial registration is to align the strip with the target image to improve the efficiency of the non-rigid registration that follows. This initial registration can take several forms. In one embodiment, the initial registration process comprises of a translational transformation to roughly align the strip with the target image. In another embodiment, the initial registration process comprises of a rigid registration that is based upon keypoint identification. In another embodiment, the initial registration process comprises of a combination of the previous two embodiments.

The strip is then registered to the target image using a novel non-rigid registration by pixel-wise local neighborhood matching (420). In the template embodiment, the target image is a template image. In the template-less embodiment, the target image is another strip. At this stage in the process, the transformed strip is assumed to be fully registered, and is set aside in a data structure until all remaining strips are fully registered. The algorithm then checks if there are any remaining strips to be registered (425). If there are, the process is repeated on the next strip. If there are not, the registered strips are averaged together (430). This produces a final image, and the algorithm is completed (435).

In the template embodiment of the invention, the target image is the template image, and it remains the template image for every iteration of the process. In this embodiment, the averaging process may or may not include the target image in the averaging calculation.

In the template-less embodiment, the target image may change with every iteration of the process. In one such variant of the template-less embodiment, the target image of the first iteration is equal to the strip with the largest area. In other variants, the initial target image may be chosen based on a different metric. The strip to be registered is then automatically chosen to be the strip with the largest region of overlap with the target image. Once the strip is fully registered by transformation, the transformed strip is set aside in a data structure, and a copy of the transformed strip is averaged with the target image to produce the new target image for the subsequent iteration of the process. In the template-less embodiment, the averaging process may or may not include the target image in the averaging calculation. In another variant of the template-less embodiment, the target image may not change with every iteration of the process, if the initial target image is sufficiently large to be the sole target of registration for the remaining strips.

FIG. 5 is a simplified illustration of both the template and template-less embodiments, showing four types of images that are used and created in the algorithm. The target image (500) can be a template image or a strip. The images to be registered (505) are subjected to an image processing step that removes motion (e.g. microsaccades) artefacts, producing motion-free strips (510). These strips are then registered to the target image and averaged together to produce the final image or images (515).

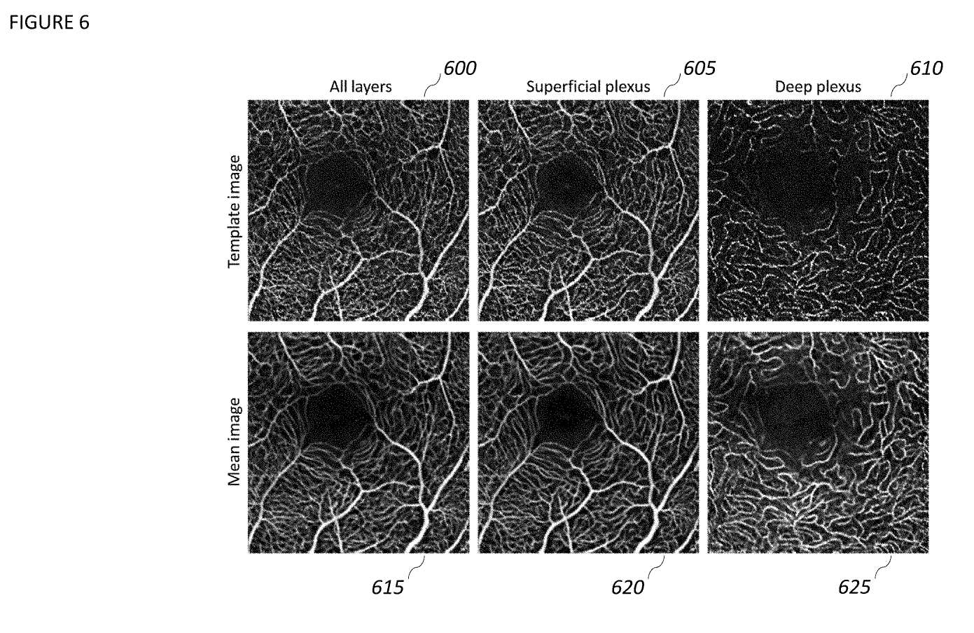

FIG. 6 is an illustration of the results of a possible variant of the template embodiment. In one embodiment of the invention, 10 serially acquired volumes centered at the foveal avascular zone (FAZ) are obtained from an eye in ˜32 s. In this embodiment, the volumes are divided into motion-free (or microsaccade-free) en face angiogram strips, which are affine registered using keypoints, followed by non-rigid registration by pixel-wise local neighborhood matching. The resulting motion corrected averaged images are presented with all of the retinal layers combined, as well as with the superficial and deep plexus layers separately. The contrast-to-noise ratio and signal-to-noise ratio of the angiograms with all retinal layers (reported as average ±standard deviation) increased from 0.52±0.22 and 19.58±4.04 dB for a single image to 0.77±0.25 and 25.05±4.73 dB , respectively, for the serially acquired images after motion correction and averaging. The improved visualization of the capillaries can enable robust quantification and study of minute changes in retinal microvasculature. In this variant, the invention was applied to an image set that contained three subsets of images: the superficial capillary layer of the retina (605), the deep capillary layer of the retina (610), and the deep and superficial layers of the retina combined (600). In this variant, the averaging process of the algorithm used a mean calculation. Thus, there are three output images: a mean image of the superficial layer (620), a mean image of the deep layer (625), and a mean image of the deep and superficial layers combined (615). The contrast of the vessels contained in the mean images is significantly improved when compared to the template images.

Both the template and template-less embodiments produce motion-free (e.g. microsaccade-free) strips from the images that are to be registered. This is accomplished by automatically identifying areas of lost fixation, which appear as white strips in the en face images. These areas are removed entirely and the regions between the stripes are preserved, thus creating the motion-free (e.g. microsaccade-free) strips.

In all embodiments, the width of a strip must exceed a predefined threshold to be considered for registration. This threshold could be equal to zero, meaning that strips of any width are accepted, or it could be equal to a non-zero value. In one embodiment, the threshold is equal to 40 pixels, as strips less than 40 pixels in width often contain large drift artefact, which has a negative effect on the registration process and output image quality.

The initial registration process used in the template and template-less embodiments (415) is used to roughly align a strip with a target image. Before initial registration, a strip is zero-padded to match the size of the target image if they are not already the same size. This initial registration can comprise of one or more forms of registration. One embodiment of the initial registration comprises of aligning a strip with the target image using a cross-correlation method. Another embodiment of the initial registration comprises of using keypoint matching to estimate a transformation that rigidly registers a strip to the target image. Another embodiment comprises of the previous two embodiments combined in any order.

Keypoint matching can be used as a component of the initial registration. Put simply, keypoints are features of interest identified in an image. Matching keypoints are two different keypoints in two different images that are identified as describing the same feature of interest. In one embodiment, the keypoint identification algorithm used is the Scale Invariant Feature Transform (SIFT) algorithm. In another embodiment, the Speeded-Up Robust Features (SURF) algorithm is used. A mathematical expression of a keypoint is a location of local scale-space extrema in the difference-of-Gaussian function convolved with the image. Further refinement to keypoints can be made by assigning each keypoint an orientation to achieve invariance to image rotation. A local image descriptor is assigned to each keypoint using the image location, scale, and orientation as found above. As the SIFT feature descriptor is invariant to uniform scaling and orientation, it is useful for identifying matching keypoints in noisy or speckled images, such as OCT-A angiograms. The calculation of Euclidean distances can be computationally expensive, and therefore matching keypoints between a target image and strip are identified as the closest corresponding keypoints by a small angle approximation to the Euclidean distance. In one embodiment, keypoints are considered matching if the ratio of the vector angles from the nearest to the second nearest match was less than a threshold value of 0.75. In one embodiment, a second check is included to ensure that the matched keypoints are no more than some maximum threshold of pixels distant in the x- or y-direction. In one embodiment, this threshold is 40 pixels. If keypoint matching is used as part or all of the initial registration, the matching keypoints are used to estimate a rigid transformation. The type of transformation used for the rigid registration may vary. The number of required keypoint matches between the strip and target image is dependent on the type of transformation desired. In one embodiment, the transformation is an affine transformation and requires a minimum of four keypoint matches. In this embodiment, strips that have a minimum of four matched keypoints are transformed using an affine transform that is estimated using the matching keypoints as inputs to an estimate geometric transform function. This function iteratively compares an affine transformation using three randomly selected keypoints, where the transformation with the smaller distance metric calculated using the M-estimator sample consensus algorithm is used as the transformation matrix for the next comparison. In one embodiment, the maximum number of random trials for finding the inliers is set to 5,000 for improved robustness.

The white lines in the target image mark the image discontinuities due to motion (e.g. microsaccades). The initial registration brings the strips from the target image into close proximity to the corresponding location in the template image. However, localized mismatch still remains in the coarsely aligned images after the initial registration step. The next step in the algorithm is to compensate for the smoother tremor and drift motions, represented by image warping and distortion, by using a novel registration method that performs non-rigid registration by pixel-wise local neighborhood matching. In one embodiment, prior to non-rigid registration, a 2×2 (or an arbitrarily chosen size) averaging filter is applied to both the target image and the aligned strip to smooth any fine speckle that may affect the non-rigid registration. In other embodiments, the size of this averaging filter could be greater than 2×2 (or a number of neighborhood pixels), or the averaging filter could not be used at all. The target image and aligned strips are then both zero padded by a certain number of pixels along each dimension of the images. In one embodiment, the amount of zero padding is equal to 15 pixels.

For each pair of coarsely registered pixels in the target strip and the template, a local neighborhood is defined on the target, and a corresponding search region is defined on the template. The purpose of the fine registration is to identify the best matching location for the target pixel in the template coordinate system based on local neighborhood matching. The size of the local neighborhood in the template image is determined by the size of features in the image. In one embodiment, the local neighborhood in the target strip is a patch with dimensions 15×15 pixels with the target pixel in the center. The best corresponding location for the target pixel (in the center of the patch) is determined using a similarity calculation of the patch against the search region in the template. Because the target strip and template are already coarsely aligned from the previous step, the search region on the template can be restricted. In one embodiment, the search region on the template is 29×29 pixels.

In one embodiment, the similarity calculation is performed using normalized cross-correlation, defined according to the formula (Mathematical Formula 11) below,

$$[ Mathematical Formula 11 ]$$

$${xcorr}_{norm} ( s , t ) = \frac{{Σ}_{x , y} [ f ( x , y ) - \overline{{f}_{s , t}} ] [ m ( x - s , y - t ) - \overline{m} ]}{\sqrt{{{Σ}_{x , y} [ f ( x , y ) - \overline{{f}_{s , t}} ]}^{2} {{Σ}_{x , y} [ m ( x - s , y - t ) - \overline{m} ]}^{2}}}$$

where, in one embodiment, m is the 15×15 pixel mask matrix centered on the selected pixel of the target strip, m̅ is the mean of the matrix m, f (x, y) is the 29×29 pixel matrix field centered on the (x, y) pixel of the template image, and f̅_̅{̅s̅,̅t̅}̅ is the mean of the template image region under the mask. In another embodiment, a normalized convolution could be used where m(x+s,y+t) are used in Formula 11 instead of m(x−s, y−t). The pixel located at the index of the maximum normalized cross-correlation is then used as the registered pixel for the strip. In other embodiments, the search region on the template image could have a size other than 29×29, and the pixel mask centered on the selected pixel of the target strip could have a size other than 15×15. The similarity calculation is repeated for rotations of the patch in the target strip. In one embodiment, the rotations are −15, −10, −5, 5, 10, and 15 degrees. In other embodiments, the similarity calculation between the target patch and the search region could be determined using a difference calculation, mean square error, or similar comparative mathematical function such as any arbitrary distance function, in place of or in addition to the normalized cross-correlation.

Once the non-rigid registration by pixel-wise local neighborhood matching is complete, the strip is fully registered. The fully registered strip is set aside in a data structure, and the process is repeated on the next unregistered strip. Once there are no unregistered strips remaining, the averaging calculation is performed on the set of fully registered strips. The number of output images produced from the averaging process is equal to the number of averaging methods used multiplied by the number of image subsets present in the overall image set. In one embodiment, these image subsets represent distinct layers of the retina.

In the embodiment of the invention represented by FIG. 4, the algorithm operates sequentially, applying a series of transformations to each strip one strip at a time. However, in other embodiments, these transformations may be applied to a set of strips in parallel. For example, in one variation of the template embodiment, all strips undergo initial registration as a set, and then this set of initially registered strips undergoes the non-rigid registration process as a set, where the target image of both registration processes is a template image.

### INDUSTRIAL APPLICATIONS

OCDR, OCT, or OCT-A, or capillary-detection systems, and methods of this instant application is very useful for diagnosis and management of ophthalmic diseases such as retinal diseases and glaucoma etc. Instant innovative OCDR, OCT, or OCT-A, or vessels-detection diagnostic systems leverage advancements in cross technological platforms. This enables us to supply the global market a valuable automated blood-vessel imaging and detection tool, which would be accessible to general physicians, surgeons, intensive-care-unit-personnel, ophthalmologists, optometrists, and other health personnel.

This device can also be used for industrial metrology applications for detecting depth-dependent flow and micron-scale resolution thicknesses.

It is to be understood that the embodiments described herein can be implemented in hardware, software or a combination thereof. For a hardware implementation, the embodiments (or modules thereof) can be implemented within one or more application specific integrated circuits (ASICs), mixed signal circuits, digital signal processors (DSPs), digital signal processing devices (DSPDs), programmable logic devices (PLDs), field programmable gate arrays (FPGAs), processors, graphical processing units (GPU), controllers, micro-controllers, microprocessors and/or other electronic units designed to perform the functions described herein, or a combination thereof.

When the embodiments (or partial embodiments) are implemented in software, firmware, middleware or microcode, program code or code segments, they can be stored in a machine-readable medium (or a computer-readable medium), such as a storage component. A code segment can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents.

What is claimed is:

1. A system, comprising:
a light source to emit a light to a beam splitter, which separates the light into two optical arms, a sample arm and a reference arm;
the sample arm further comprises of a sample and light delivery optics; and
a reference arm comprising a reference mirror;
a light returning from the sample and reference arms combined through the beam splitter and directed towards at least one detector to generate an optical interference signal;
an instrument controller for controlling the acquisition of the optical interference signal;
a processor to process the interference signal to generate at least one image;
at least one controllable wavefront modifying optical element to modify the wavefront arriving from the sample;
a calibration signal to stabilize a phase of the optical interference signal.

2. The system of claim 1; where the processor generates images comprising flowing material.

3. The system of claim 1; where the processor generates angiograms.

4. The system of claim 1; where the features of interest comprise of at least one of capillaries and vessels.

5. The system of claim 1; wherein the light source is a swept-source.

6. The system of claim 1; where the detection arm further comprises of a spectrometer.

7. The system of claim 1; wherein phase stabilization is performed with a calibration signal derived from a calibration interferometer.

8. The system of claim 1; wherein the optical interference signal is generated using bidirectional scans.

9. A method, comprising:
sending light through a sample arm to a sample; the sample arm having at least one controllable wavefront modifying optical element;
acquiring an optical interference signal of a sample by a detector;
assembling the optical interference signal into images;
determining merit functions of the images; and
adjusting the wavefront modifying element; and
visualizing at least one of blood vessels, capillaries, lymph vessels in angiograms.

10. The method of claim 9, wherein the optical interference signal is processed to generate an A-scan;
and an OCT B-scan is generated by acquiring a plurality of A-scans;
and a plurality of B-scans are collected at the same location to generate BM-scans.

11. The method of claim 10, wherein controlling a scanning mechanism in the sample arm is used to acquire three dimensional volumetric data of the sample.

12. The method of claim 10; where the processing is implemented in at least one of FPGA, DSP and application-specific-integrated-circuits.

13. The method of claim 10; where the bidirectional scans are performed to generate BM-scans.

14. The method of claim 10, wherein the angiograms are obtained by monitoring variations of the optical interference signal.

15. The system of claim 10, wherein the optical interference signal is generated using a stepped bidirectional scan.

16. A system, comprising:
a light source to emit a light to a beam splitter, which separates the light into two optical arms, a sample arm and a reference arm;
the sample arm further comprises of a sample and light delivery optics; and
a reference arm comprising a reference mirror;
a light returning from the sample and reference arms combined through the beam splitter and directed towards at least one detector to generate an optical interference signal;
an instrument controller for controlling the acquisition of the interference signal;
a processor to process the interference signal to generate at least one image; and
the processor generates images comprising flowing material;
and strip-based registration is performed.

17. The system of claim 16; where the images are divided into target images strips.

18. The system of claim 17; where the target images are registered to a template image using pixel-wise local neighborhood matching.

19. The system of claim 16, where the angiogram is obtained using monitoring variations in at least one of image intensity and the phase of the optical interference signal.

20. The system of claim 16, wherein a template is iteratively generated by mosaicking target strips.
